(19) United States
(12) Patent Application Publication
(10) Pub. No.: US 2016/0206623 A9
(43) Pub. Date: Jul. 21, 2016

(54) Composition For Prevention of Vasoactivity in the Treatment of Blood Loss and Anemia

(71) Applicant: Jan BLUMENSTEIN, Toronto (CA)

(72) Inventor: Jan BLUMENSTEIN, Toronto (CA)

(21) Appl. No.: 14/252,318

(22) Filed: Apr. 14, 2014

Related U.S. Application Data
(62) Division of application No. 13/796,110, filed on Mar. 12, 2013, now abandoned.
(60) Provisional application No. 61/622,612, filed on Apr. 11, 2012.
(60) Provisional application No. 61/622,615, filed on Apr. 11, 2012.

(51) Int. Cl. A61K 31/554; A61K 45/06; A61K 31/517; A61K 31/519; A61K 31/53

(52) U.S. Cl. CPC A61K 31/554; A61K 31/519; A61K 31/53; A61K 31/517; A61K 45/06

(57) ABSTRACT

The present invention relates to the prevention of cardiovascular and central nervous system side effects in mammals who receive transfusions of hemoglobin based oxygen carriers (HBOC) or stored blood products containing a concentration of hemoglobin sufficient to induce vasoconstriction, by adding a vasoactivity reducing effective amount of one or more phosphodiesterase inhibitors in combination with a calcium channel blocker and/or an alpha agonist, to the circulation, or alternatively to the HBOC or stored blood, thereby preventing the manifestation of vasoactivity attributable to the presence of free tetrameric hemoglobin (Hb).

### CROSS-REFERENCE TO RELATED APPLICATIONS

This application is a divisional application of co-pending application Ser. No. 13/796,110, filed on Mar. 12, 2013, and claims benefit of priority to U.S. Provisional Patent Application Ser. No. 61/622,612, filed Apr. 11, 2012, and to U.S. Provisional Patent Application Ser. No. 61/622,615, filed Apr. 11, 2012, the contents of which are incorporated herein by reference in their entireties.

### FIELD OF THE INVENTION

This invention relates to a composition and methodology for reducing or preventing vasoactivity which occurs as a result of the introduction of therapeutic agents into the circulation directly, and particularly relates to the use of phosphodiesterase (PDE) inhibitors, in combination with additional agents, which act synergistically to prevent or reduce vasoactivity, which occurs concomitant to the addition of free hemoglobin and hemoglobin based oxygen carriers (HBOC) to the circulation, or alternatively, due to the presence of free tetrameric hemoglobin (Hb) in stored donated blood, as a result of hemolysis.

### BACKGROUND OF THE INVENTION

Prior to the use of blood for transfusion, it is a requirement to type and cross-match the blood to minimize the risk of transfusion reactions. Basic type and cross-matching require several minutes to accomplish, and a complete type and cross-match can take up to an hour. Furthermore, the risk of HIV transmission has been estimated to be 1 in 500,000 units of blood and the risk of Hepatitis C transmission has been estimated to be 1 in 3,000 units. The safety of blood supplies and blood logistics are critical issues in developing countries, where the risk of infectious disease transmission, as well as the risk of outdated supply, is relatively higher. Therefore, there is a need to find blood substitutes or artificial blood compositions that avoid disease transmission and provide rapid response to improve chances of survival.

In clinical settings, artificial blood use is necessary for volume expansion and oxygen therapeutics. Volume expander agents are generally inert, merely increasing blood volume, and thereby allowing the heart to pump fluid efficiently. Oxygen therapeutics are designed to mimic human blood's oxygen transport ability. Oxygen therapeutics can be divided in two categories based on transport mechanism: perfluorocarbon based, which function by simple dissolution of oxygen, and hemoglobin protein based, which transports oxygen by facilitated capture and release. In hemoglobin-based products, pure tetrameric hemoglobin (Hb) separated from red blood cells (RBCs) may not be useful for a number of reasons, including low molecular weight, instability, induction of renal toxicity, and unsuitable oxygen transport and delivery characteristics when separated from red blood cells.

Desirable characteristics of hemoglobin based oxygen delivery therapeutics are: toxicity-free, lack of induction of harmful immunogenic response, satisfactory oxygen carrying and delivery capacity, suitable circulatory persistence to permit effective oxygenation of tissues, long shelf life, capacity for storage at room temperature, absence of viral or other pathogens to prevent disease transmission, elimination of the requirement for blood typing, and capacity for deployment by first responders, such as paramedics, front line military medics, etc. These characteristics provide a rapid, safe response to blood loss and the immediate support of tissue metabolic needs, thus improving the chances for survival.

Unfortunately, hemoglobin based oxygen therapeutics have been shown to exert various degrees of vasoactive effects both in animal and human studies (Winslow et al., Adv Drug Del Rev 2000; 40: 131-42; Stowell et al., Transfusion 2001; 41: 287-99; Spahn et al., News Physiol Sci 2001; 16: 38-41; Spahn et al., Anesth Analg 1994; 78: 1000-21; Kasper et al., Anesth Analg 1996; 83: 921-7; Kasper et al., Anesth Analg 1998; 87: 284-91; Levy et al., J Thorac Cardiovasc Surg 2002; 124: 35-42;). Vasoactivity may be due to the effects of these products in binding extracellular NO (Kasper et al., Anesth Analg 1996; 83: 921-7; Dietz et al., Anesth Analg 1997; 85: 265-273; Schechter et al., N Engl J Med 2003; 348: 1483-5), endothelial release (Gulati et al., Crit. Care Med 1996; 24: 137-47), or sensitization of peripheral α-adrenergic receptors (Gulati et al., J Lab Clin Med 1994; 124: 125-33). Alternatively, the increased vasoconstrictive effects could be due to increases in the rate of oxygen release, secondary to the administration of these products, at a higher concentration than RBCS, resulting in vasoconstriction (Winslow et al., J Intern Med 2003; 253: 508-17; McCarthy et al., Biophys Chem 2001; 92: 103-17; Intaglietta et al., Cardiovasc Res 1996; 32: 632-43; Vandegriff et al., Transfusion 2003; 43: 509-16).

It has also been well documented that stored donated blood undergoes hemolysis. The extent of the hemolysis depends on a variety of factors: the donor, the method of collection, the nature and length of storage and the method of administration. There have been many attempts at using additives to prevent hemolysis without much success. This invention further deals with prevention of the consequences of hemolysis.

The tendency for stroma-free Hb solutions to induce blood pressure increases has been known. It has been demonstrated that some cross-linked Hb solutions could increase mean arterial pressure as much as 25-30% in a dose-dependent manner within 15 minutes of administration and that the effect could last as long as 5 hours.

Vasoconstriction may be due to NO scavenging by hemoglobin based therapeutics (Katsuyama et al., Artif Cells Blood Substit Immobil Biotechnol 1994; 22:1-7; Schultz et al., J Lab Clin Med 1993; 122:301-308). Vasoconstriction could also be caused by the contamination of the hemoglobin by phospholipids and endotoxins.

NO is a smooth-muscle relaxant that functions via activation of guanylate cyclase and the production of cGMP, or by direct activation of calcium-dependent potassium channels. The increase in the free Hb can result in an increase in the NO binding. The increase in the NO binding can result in transient, and in repeat dosing, sustained, hemodynamic changes responding to vasoactive substances or the lack of vasoactive regulatory substances. In some circumstances the lack of nitric oxide may lead to blood pressure increases and if prolonged, hypertension. It has been demonstrated that NO may bind to the reactive sulfhydryls of Hb and may be transported to and from the tissues in a manner analogous to the transport of oxygen by heme groups (Jia et al., Nature 1996; 80:221-226).

Nitric oxide along with precapillary sphincter movement are regulators of the arteriolar perfusion of any tissue. Nitric oxide is synthesized and released by the endothelium in the arterial wall, where it can be bound by hemoglobin in red blood cells. When a tissue is receiving high levels of oxygen, nitric oxide is not released and the arterial wall muscle contracts making the vessel diameter smaller, thus decreasing perfusion rate and causing a change in cardiac output. When demand for oxygen increases, the endothelium releases nitric oxide, which causes vasodilatation. The nitric oxide control of arterial perfusion operates over the distance that NO diffuses after release from the endothelium. Nitric oxide is also needed to mediate certain inflammatory responses. For example, nitric oxide produced by the endothelium inhibits platelet aggregation. Consequently, as nitric oxide is bound by cell-free hemoglobin, platelet aggregation may be increased. As platelets aggregate, they release potent vasoconstrictor compounds such as thromboxane A₂ and serotonin. These compounds may act synergistically with the reduced nitric oxide levels caused by hemoglobin scavenging, resulting in significant vasoconstriction. In addition to inhibiting platelet aggregation, nitric oxide also inhibits neutrophil attachment to cell walls, which in turn may lead to cell wall damage. Because nitric oxide binds to hemoglobin inside the red blood cell, it is expected that nitric oxide may bind to free Hb (stroma free cross-linked tetrameric Hb) as well.

In many formulations free Hb and stabilized hemoglobin infusions appear to be linked to vasoconstriction of the blood vessels, resulting in extremely high blood pressures. The hemoglobin moiety of these products can diffuse into the endothelial lining of the vascular wall and act to bind and remove NO which is needed for maintaining the normal tone of the vascular wall, thereby resulting in vasoconstriction of the smooth muscle cells of the vascular wall. Therefore, it is important to minimize the impact of administration of most free Hb on the arterial system during administration.

Vasoactive agents such as verapamil, atenocard, sildenafil citrate, etc., may be administered to the patient prior to free Hb infusion. This is intended to ensure that the arterial system is minimally changed during infusion. Nitric oxide and verapamil are preferred vasoactive agents. Slow channel calcium blockers (or a selective inhibitor of cyclic guanosine monophosphate (cGMP)-specific phosphodiesterase type 5 (PDE5), such as sildenafil citrate) may also be helpful in the prevention of the severe vasoconstriction. However, a slower infusion rate may not be possible with respect to a trauma patient when demand for volume is acute and critical.

There are three intracellular factors that result in vasodilation of blood vessels. These are related to calcium transport across cell membranes, adrenergic stimulation (cAMP mediated), and endothelium-derived relaxing factors (nitric oxide, cGMP mediated).

The most significant mechanism of regulation of vasoactivity is governed by the relationship of nitric oxide (NO) and hemoglobin in the red blood cell. Endothelial cells contribute to the control of local vascular tone by formation of NO. Since there is excess of NO, its concentration is regulated by intra-cellular formation of S-nitrosohemoglobin (SNO-Hb). SNO-Hb is a vasodilator whose activity is allosterically modulated by oxygen. The allosteric modulation depends on intracellular redox mechanism and at low oxygen tension the SNO-Hb produces vasodilatation.

In case of tetrameric HB or HBOCs in the circulation, in the extra cellular form, there is no intracellular mechanism to provide for the allosteric modulation. As a consequence, due to the lack of the presence of SNO-Hb in the proper allosteric form, vasoconstriction is the predominant feature. The necessity for regulation of vasoactivity then has to rely on modulating the vascular smooth muscles of the arterioles in order to achieve vasodilatation. In accordance with this invention reducing vasoactivity will be understood to include reducing or eliminating the vasoconstriction initiated by the administration of HBOCs or tetrameric Hb to the circulation of a mammal.

The role of NO in the vasodilation through the cGMP pathway is as follows:
cGMP is formed from guanosine triphosphate by the enzyme guanylate cyclase (sGC, soluble guanylate cyclase);
sGC activity is increased 400 fold by NO, thus cGMP is available for smooth muscle relaxant activity;
cGMP increases the activity of the myosin light chain phosphatase, producing dephosphorylation and smooth muscle relaxation;
cGMP is degraded by numerous phosphodiesterases (PDE) the product being 5′-GMP.

The present inventors have now discovered that the introduction of vasoactivity reducing effective amounts of one or more PDE inhibitors, in combination with one or more calcium channel blockers or alpha agonists, will result in vasodilatation, thereby reducing or preventing vasoactivity otherwise induced by HBOC products, or from stored whole blood or stored whole blood products, hereinafter referred to as “stored blood products”, having vasoactivity inducing concentrations of free tetrameric hemoglobin.

### PRIOR ART

U.S. Pat. No. 4,994,367 to Bode, et al. is directed toward extending the shelf life of platelet preparations. This is accomplished by producing platelet-rich plasma (PRP) from whole blood, adding a platelet activation inhibitor thereto, centrifuging the PRP to deposit the platelets on the bottom of the centrifuge container, removing the platelet-free plasma supernatant therefrom and adding a plasma-free liquid platelet storage medium thereto. A preferred platelet activation inhibitor for the process comprises an adenylate cyclase stimulator in combination with a phosphodiesterase inhibitor. A preferred adenylate cyclase stimulator is Prostaglandin El, a preferred phosphodiesterase inhibitor is Theophylline, a preferred plasmin inhibitor is Aprotinin, and a preferred thrombin inhibitor is N-(2-naphthylsulfonylglycyl)-D,L-amidinophenylalaninpiperidide.

International Publication WO/2008/063868 to Zapol et al is directed toward compositions and methods for preventing or reducing vasoconstriction in a mammal following administration of a vasoactive oxygen carrier (e.g., a heme-based oxygen carrier such as a hemoglobin-based oxygen carrier). The methods include administering to a mammal a composition containing a vasoactive oxygen carrier in combination with one or more of a nitric oxide-releasing compound, a therapeutic gas containing gaseous nitric oxide, a phosphodiesterase inhibitor, and/or a soluble guanylate cyclase sensitizer. Zapol et al discuss use of a nitric oxide gas as an inhibitor of vasoactivity and demonstrate such utility. Zapol et al goes on to allege utility for nitric oxide followed by PDE inhibitor administration, as well as the use of a PDE inhibitor alone, but does not have an enabling disclosure for practicing the in vivo dosing of PDEs to a mammal in order to prevent or reduce vasoactivity of HBOC compositions administered thereto. Zapol et al fails to teach or suggest the synergistic result achieved, in vivo, by the instant invention in combining sub-optimal doses of a PDE inhibitor, calcium channel blocker and/or an alpha agonist in order to minimize or totally mitigate the vasoconstriction evoked by the use of HBOCs or stored whole blood.

What is therefore still lacking in the art is a composition and method for its use designed to eliminate the vasoactive risk concomitant with the administration to patients of a heme-based oxygen carrier such as a hemoglobin-based oxygen carrier or aged stored whole blood, which has undergone sufficient hemolysis to induce vasoactivity.

### SUMMARY OF THE INVENTION

In accordance with the present invention there is provided a new composition and method for reducing or preventing vasoactivity and the cardiac and hypertensive problems associated therewith, subsequent to intravenous introduction of blood substitutes, such as HBOCs, as well as stored transfusion material containing a vasoactivity inducing concentration of hemolysed red blood cells.

In accordance with the present invention, vasoactivity reducing effective amounts of certain phosphodiesterase inhibitors, in combination with one or more calcium channel blockers and/or alpha agonists, are taught for preventing vasoactivity subsequent to introduction of HBOCs, stored blood, or other intravenous agents.

Accordingly, it is a primary objective of the instant invention to provide a vasoactivity neutralizing composition and methodology for administering a combination of one or more PDE inhibitors, singly or in some combination, along with one or more calcium channel blockers and/or alpha agonists, at amounts effective to reduce or prevent the vasoactivity initiated by the exposure of the circulatory system of a mammal to a hemoglobin-based oxygen carrier, or stored blood products containing free hemoglobin in amounts sufficient to induce vasoactivity, thereby preventing cardiovascular and central nervous system sequele. It is understood that the aforementioned administration of said one or more vasoactivity neutralizing compositions may be by way of dosing the HBOCs or stored blood directly, or administration thereof into the main circulation in an amount effective for regulating the degree of vasoactivity.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### ABBREVIATIONS

RTA—Rat Thoracic Aorta segment

NO—Nitric Oxide

PHE—Phenylephrine

SFH—Stroma Free Hemoglobin

Hb21—Sildenafil Citrate (Viagra) (PDE inhibitor)

DLT—Diltiazam (Calcium channel blocker)

HES—Hexaethylstarch Hemoglobin (HBOC)

HBOC—Hemoglobin Based Oxygen Carrier

TER—Terazosin (alpha agonist blocker)

VAR—Vardenafil (Levitra) (PDE inhibitor)

HDL—combination of Hb21 and DLT

PKA, PKC, PKG—phosphokinases

### BRIEF DESCRIPTION OF THE FIGURES

FIGS. 1A and 1B illustrate the vasoactivity of segments of the rat thoracic aorta when the agonist phenylephrine(PHE) was added to the tissue chamber before or after the heme containing molecule. FIG. 1A illustrates addition using stroma free hemoglobin, while FIG. 1B illustrates addition of hexaethylstarch hemoglobin (HES);

FIG. 2 illustrates the dose response of segments of the rat thoracic aorta when exposed to different concentrations of the agonist (PHE), (a) 0.25×10⁻⁷ M, (b) 0.5×10⁻⁷ M, (c) 10⁻⁷ M;

FIGS. 3A and 3B show comparative tracings, which further shows the difference in vasoactivity when PHE (FIG. 3A) versus SFH (FIG. 3B), is added first to the tissue chambers;

FIG. 4 illustrates the vasoactivity of segments of the rat thoracic aorta treated with stroma free hemoglobin (0.44 uM) and phenylephrine (PHE) which have been pretreated with sildenafil citrate at concentrations of (a) 0, (b) 10⁻⁶ M, (c) 10⁻⁵ M, (d) 10⁻⁴ M;

FIG. 5 illustrates the changes in vasoactivity when sildenafil (10⁻⁴ M) was added (b) to the SFH+PHE pre contracted segments of the rat thoracic aorta (a);

FIG. 6 shows the reduction of vasoactivity of the segments of the rat thoracic aorta, exposed to SFH+PHE, when pretreated with suboptimal doses of (a) no additives, (b) sildenafil citrate, (c) diltiazam, and (d) both sildenafil citrate and diltiazam;

FIG. 7 illustrates the effect of combining suboptimal concentrations of sildenafil citrate and DLT on the vasoactivity of segments of the rat thoracic aorta. The bar graphs represent the following: (a) PHE, (b) SFH+PHE, (c) DLT+PHE, (d) sildenafil citrate+PHE, (e) sildenafil citrate+DLT+PHE, (f) sildenafil citrate+DLT+SFH+PHE;

FIG. 8 illustrates similar experiments performed with hexaethylstarch hemoglobin (HES). The results indicate that the calcium channel blocker (DLT) reduces the vasoconstrictive effect of PHE (a1, 0; b1, 10⁻⁷M; c1, 10⁻⁶M; d1, 10⁻⁵M). Similarly when HES (4.4 uM) is added to the tissue chambers with the same concentration of additives as in series (a), (see a2, b2, c2, d2) the vasoconstriction is reduced. Adding sildenafil citrate (10⁻⁵ M) to the tissue chambers, represented by e1 and e2, having the same additives as d1 and d2, the vasoconstriction is totally abolished;

FIG. 9 illustrates a similar experiment conducted with an alpha agonist blocker, terazosin. The results indicate that terazosin in suboptimal concentration (10-8 M) reduces the vasoconstrictive effect of SFH. In combination with sildenafil citrate the vasoconstriction is reduced significantly. The bar graphs represent the following additives: (a) SFH+PHE, (b) TER+SFH+PHE, (c) TER+sildenafil citrate (10⁻⁵M)+SFH+PHE, (d) TER+sildenafil citrate (2×10⁻⁵M)+SFH+PHE;

FIG. 10 illustrates the reduction of vasoconstriction by vardenafil and terazosin. In this graph HES (0.44 uM) was used in all the tissue chambers. The following additions were made in each of the four tissue chambers: (a) PHE 10⁻⁷ M, (b) VAR 0.5×10⁻⁶ M+PHE 10⁻⁷ M, (c) VAR 10⁻⁶ M+PHE 10⁻⁷ M and (d) VAR 0.5×10⁻⁶ M+TER 10⁻⁸ M+PHE 10⁻⁷ M.

### DETAILED DESCRIPTION OF THE INVENTION

In accordance with the present invention vasoactivity refers to the ability of blood vessels to expand and contract. Through vasoactivity the body controls the flow of blood through individual organs, to accommodate the variation in blood flow and regulate arterial pressure.

The current view of the vasoactivity of the smooth muscle in the arterioles is regulated by three biochemical pathways and influencing three phosphokinase enzymes, phosphokinase A—activated by the agonist; phosphokinase C—acts through calmodulin, i.e. it is calcium dependent; and phosphokinase G—activated by cGMP which in turn is a result of activation of guanylate cyclase by nitric oxide. cGMP in turn is broken down by a phosphodiesterase.

In practicing the current invention, a composition including at least one phosphodiesterase (PDE) inhibitor or combination thereof, in combination with a calcium channel blocker and/or an alpha agonist blocker, will be included in hemoglobin based oxygen carriers, or alternatively administered intravenously to a mammal who is to be infused with an HBOC, in an amount effective to eliminate or substantially reduce any demonstrable vasoconstriction caused by the HBOC as a result of its administration to an individual in need thereof.

It has been well documented that peri-operative transfusions increase the morbidity and mortality rate significantly. This effect of transfusions is even more pronounced in the elderly. Due to the cardiovascular side effects post HBOC transfusion, the FDA (USA) had stopped all clinical trials of existing HBOCs.

SFH, under certain conditions, causes constriction of the smooth muscles of the arterioles. (SFH and HBOC can be used interchangeably). Vasoactivity in the arterioles is regulated by nitric oxide (NO), a signaling molecule, synthesized in the endothelium and agonists (epinephrine and nor-epinephrine, phenylephrine, PHE). The agonists affect phosphokinases: phosphokinase A (PKA) and phosphokinase C (PKC), while NO exert its influence on the enzyme guanylyl cyclase that converts GTP to cGMP.

cGMP is another signaling molecule that acts on phosphokinase G (PKG), which relaxes smooth muscle of arterioles. cGMP is broken down by a phosphodiesterase (PDE).

In the red blood cell NO exists in an allosteric location in combination with Hb (SNO-hemoglobin). Here, with some cellular energy and depending on oxygen partial pressure, it releases or takes up NO. The perfusion through the arterioles is then regulated via the partial pressure of oxygen. SFH free in the circulation (outside red blood cell) also combines with Hb but there is no energy source to influence the homeostasis, thus NO cannot exert its signaling role on guanylyl cyclase and consequently on PKG, and vasoconstriction occurs.

The other possible mechanism is the structure of guanylyl cyclase. This molecule is also a heme protein and it is possible that that SFH acts as a competitive inhibitor in the binding of NO.

In view of the above considerations, we have examined PDE inhibitors to preserve the accumulated cGMP and perhaps some guanylyl cyclase enzyme function.

Illustrative, albeit non-limiting examples of phosphodiesterase inhibitors include: Zaprinast, 5-(2-Propoxyphenyl)-1H-[1,2,3]triazolo[4,5-d]pyrimidin-7(4H)-one, (M&B 22948; 2-o-propoxyphenyl-8-azapurine-6-one; Rhone-Poulenc Rorer, Dagenham Essex, UK); WIN 58237 (1 -cyclopentyl-3-methyl-6-(4-pyridyl)pyrazolo[3,4-d]pyrimidin-4-(5H)-one; Silver et al. (1994) J. Pharmacol. Exp. Ther. 271: 1143); SCH 48936 ((+)−6a,7,8, 9,9a, 10,11,1 la-octahydro-2,5-dimethyl-3H-pentalen (6a,1,4,5)imidazo[2,1- b]purin-4(5H)-one; Chatterjee et al. (1994) Circulation 90:1627, abstract no. 3375); KT2-734 (2-phenyl-8-ethoxycycloheptimidazole; Satake et al. (1994) Eur. J. Pharmacol. 251 :1); E4021 (sodium 1-[6-chloro-4-(3,4-methylenedioxybenzyl)-aminoquinazolin-2-y]piperidine-4 -carboxylate sesquihydrate; Saeki et al. (1995) J. Pharmacol. Exp. Ther. 272:825); sildenafil (Viagra®); tadalafil (Cialis®); vardenafil (Levitra®), avanafil, lodenafil, mirodenafil, udenafil, xanthine, caffeine, theophylline, theobromine, aminophylline, oxtriphylline, dyphylline, pentoxifylline, isobutylmethylxanthine, dipyridamole, papaverine, and mixtures thereof.

In order to reduce the required effective concentration of Hb21 or similar PDE inhibitors, and decrease the effect on vasoactivity of SFH, an additional additive was considered. One class of additive that was considered was the calcium channel blockers. These compounds reduce the intracellular calcium ion and the agonist acts through calmodulin to increase the cGMP effectiveness and produce relaxation of the smooth muscle.

Illustrative, albeit non-limiting examples of calcium channel blockers useful in the present invention include amlodipine, diltiazem, felodipine, isradipine, nifedipine, nicardipine, nimodipine, nisoidipine, verapamil, and mixtures thereof. It has also been shown that agonists exert their influence via Ca⁺⁺ and they act on phosphokinase C (PKC). PKC when activated induced contraction of the smooth muscle. For this reason a calcium channel blocker was considered to act synergistically with Hb21. In considering the adrenergic effect of PHE, diltiazam was selected as a calcium channel blocker in the instant experiments.

Alternatively, alpha agonists were considered as candidates having the possibility of acting synergistically with the PDE inhibitors. Such alpha agonists were selected from the class of adrenergic antagonists including, albeit not limited to, prazosin, terazosin, urapidil, labetalol, yohimbine, phenoxybenzamine, phentolamine, tolazoline, acebutolol, atenolol, and mixtures thereof.

For example, Terazosin, an alpha (agonist) blocker, blocks the action of agonists and it affects both PKA and PKC. In the case of PKC it prevents the vasoconstriction, and with PKA it can prevent vasoconstriction or enhance vasodilatation. Terazosin was also used in the instant experiments in conjunction with Hb21 to prevent vasoconstriction when SFH is introduced in the tissue chambers.

Both calcium channel blockers and alpha agonists have been considered in these experiments to investigate the synergistic effect and thus lower the required effective dose of the drugs.

### Method Considerations

The accepted method to study vasoactivity is to pre-constrict the RTA with epinephrine and then add the test substances to observe their effect. This is the case in studies involving SFH. The results show a 20-30% increase in vasoconstriction.

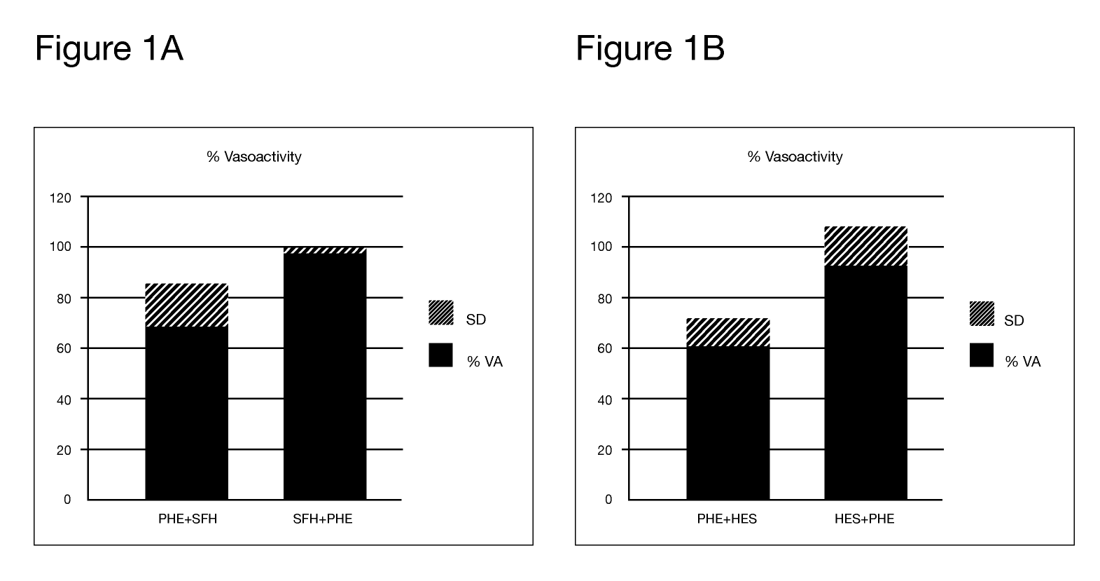

In our experiments we found that by adding SFH first and then adding PHE, SFH does not affect vasoactivity (more than 100 tests). The vasoconstriction produced by the addition of PHE after the SFH results in 30-40% increase of PHE induced contraction (FIG. 1A).

Using an HBOC, hexaethylstarch Hb (HES), we confirmed the same observation (FIG. 1B).

Once SFH is added previously to the chamber, the PHE stimulation of contraction when added without SFH raises to the level of SFH+PHE.

We have also measured the vasoactivity of PHE with different concentrations of Hb21. It seems that Hb21 and VAR both reduce the effectiveness of PHE to induce vasoconstriction.

### Materials and Methods

Chemicals were purchased from Sigma-Aldrich or Fisher Scientific Co. HB-hexaethylstarch was purchased from Therapure Biopharma Inc., Toronto, ON, Canada, as well as stroma free hemoglobin (HbA0). Sildenafil and vardenafil were obtained from Globec International, Toronto, ON, Canada.

The experiments were performed using adult male Wistar rats weighing 250-300 g. They were housed in the Animal Resources Centre of the University Health Network, Toronto (“UHN”).They were fed ad libitum on standard diet. All animal procedures were conducted as approved by the Animal Care Committee, UHN.

The animals were anesthetized with inhaled Isoflurane 99.9%. The thoracic aorta (RTA) was quickly dissected, and placed in Krebs-Heneslite buffer@ph 7.4 containing D-glucose 2.00 g, magnesium sulfate (anhydrous) 0.141 g, potassium phosphate monobasic 0.160 g, potassium chloride 0.350 g, sodium chloride 6.90 g, calcium chloride dihydrate 0.373 g, and sodium bicarbonate 2.10 g /liter). They were cleaned of surrounding tissues. The arteries were cut into rings 3-4 mm in length. The aortic segments were suspended in a four chamber Radnoti 10 ml tissue bath system by “L” glass tissue hook with a stainless steel wire for lower ring support and a triangular upper ring support connecting with a silk connection to an ADlnstruments, Force Transducer (MLT 0201). Isometric tension changes were analyzed with LabChart 7 Pro and interpreted via a dedicated laboratory iMac computer.

The bath temperature was maintained at 37.4° C. and a constant stream of 95% O₂/5% CO₂ was bubbled through the chambers and the buffer reservoir during the experiments. The buffer was changed in the tissue chambers every 15 minutes.

The arterial rings were subjected to an optimal tension of 2.5 g over a 90-minute period, this optimum was obtained from preliminary experiments. The phenylephrine pre-contracted rings were exposed to acetylcholine (1 uM) to verify intact endothelium. Each measure was taken as the average of at least 4 segments and is expressed as a percentage of the original (SFH+PHE) contraction of each RTA segment.

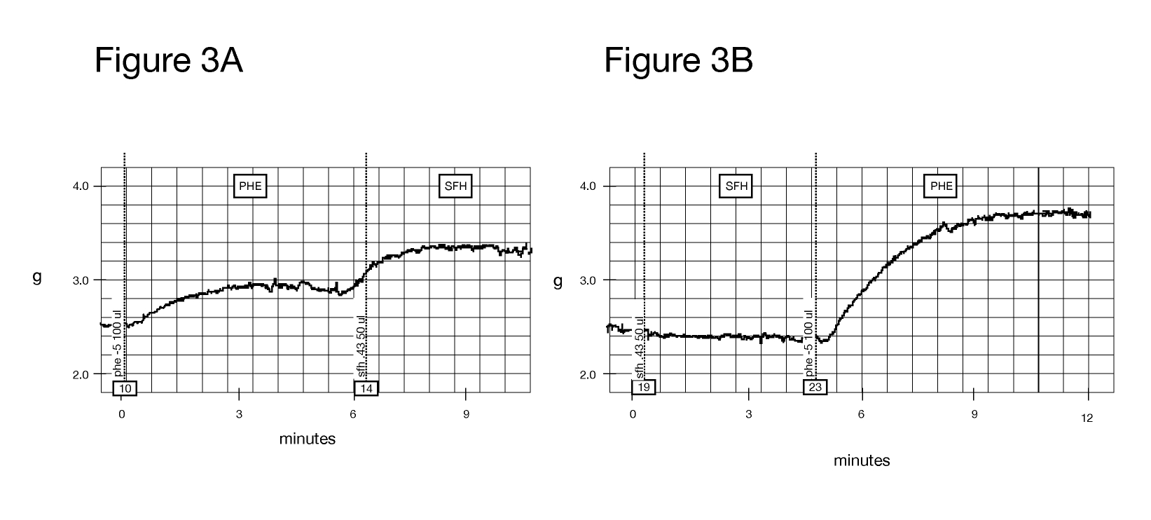

Four RTA rings were simultaneously exposed to the same treatment in individual tissue chambers, and their respective contractions were evaluated (with contraction elicited with PHE and SFH+PHE) at the beginning, during the experimentation, and at the end to test for fatigue. It has been observed during initial testing, and further illustrated in the tracings depicted in FIGS. 3A and 3B, that addition of SFH to the pre-contracted RTA was less effective than adding SFH to the chambers first and PHE subsequently to produce the vasoconstriction.

### Results

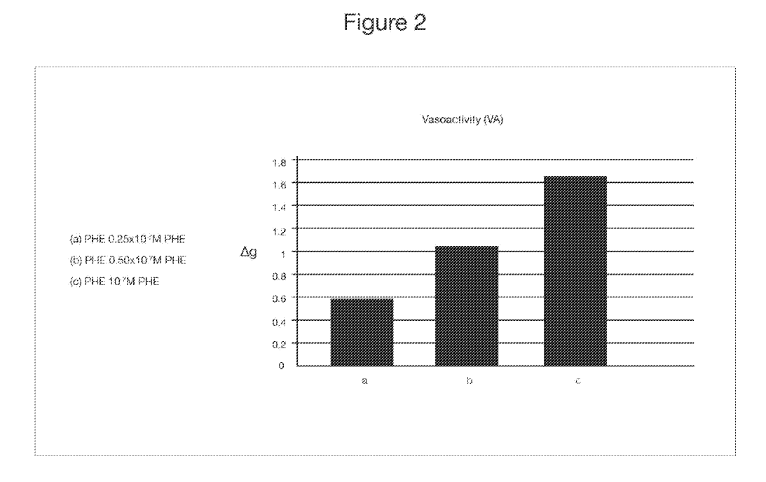

In order to determine the effective dose of PHE (the agonist) we used 3 different concentrations of the agonist. FIG. 2 illustrates the effect of increasing concentration of PHE on the vasoactivity of the RTA.

The results show the increase in tension (gm) of the RTA by adding 25 ul, 50 ul and 100 ul respectively of PHE at a concentration of 10⁻⁵ M to a 10 ml tissue chamber respectively.

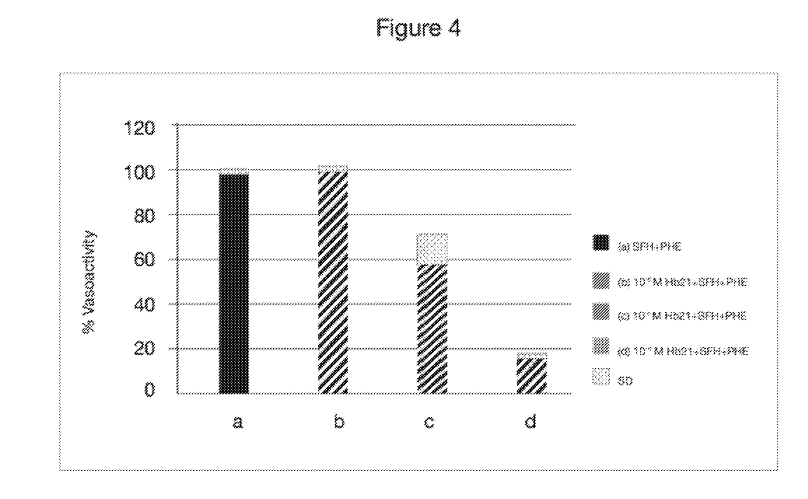

Referring to FIG. 4, it shows a typical record of contractions occurring with different additive concentrations.

With reference to FIG. 4, segments of the RTA were prepared and were equilibrated to a tension of 2.5 g. Once stabilized, in succession, Hb21, SFH and PHE were added at 5-minute intervals. SFH was added to the chambers to a concentration of 4.4 uM. The Hb21 was added in 100 ul aliquots to a final concentration of 10⁻⁶, 10⁻⁵ and 10⁻⁴ M.

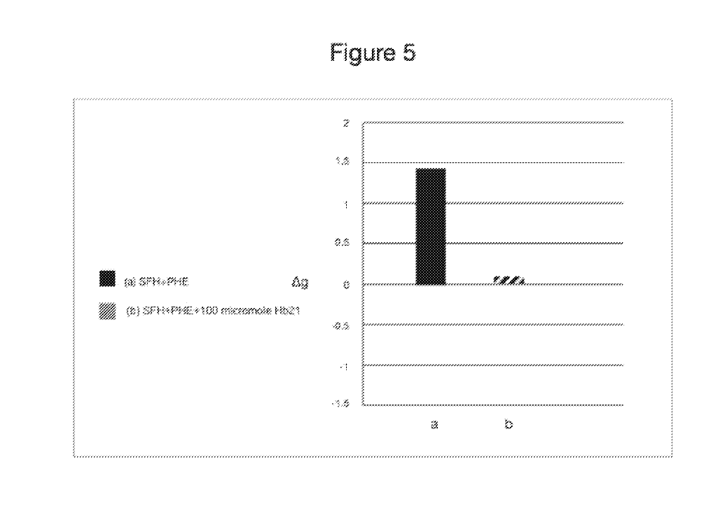

With respect to FIG. 5, the results illustrate the effect of Hb21 on the SFH+PHE pre-contracted RTA. Hb21 was added to the pre-contracted segment of the rat thoracic aorta.

In order to reduce the effective concentration of Hb21 and decrease the effect on vasoactivity of SFH inclusion of an additional additive was explored. One class of additives that was considered was the calcium channel blockers. Illustrative, albeit non-limiting examples of channel blockers useful in the present invention include amlodipine, diltiazem, felodipine, isradipine, nifedipine, nicardipine, nimodipine, nisoidipine and verapamil. In considering the adrenergic effect of PHE, diltiazam was selected as a calcium channel blocker in the instant experiments.

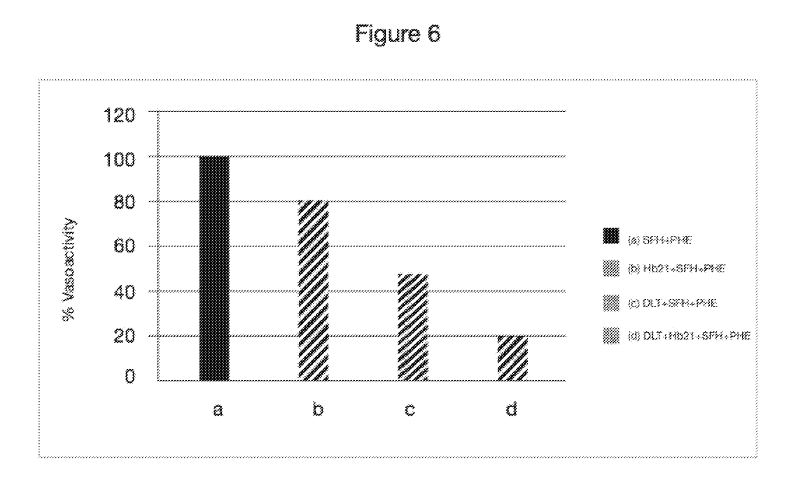

FIG. 6 shows the effect of adding diltiazam with a suboptimal concentration of Hb21. The reduction of the vasoconstriction of the segment of the rat thoracic aorta indicates a synergistic effect of Hb21 and diltiazam. Combining Hb21 to a final concentration in the tissue chamber of 10⁻⁵ M and diltiazam at a concentration of 10⁻⁵ M, produced a significant reduction of the vasoconstriction.

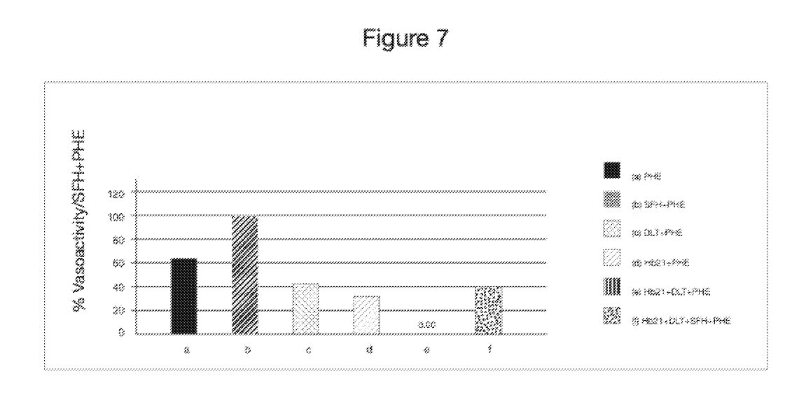

Referring to FIG. 7, this shows the suboptimal concentration of Hb21 (10⁻⁵ M) and DLT (10⁻⁵ M) reduces the vasoconstriction of segments of the rat thoracic aorta by addition of SFH (4.4×10⁻⁶ M) by 60%.

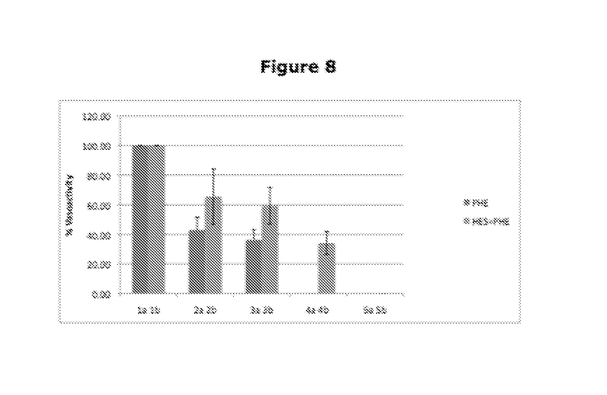

FIG. 8 illustrates similar experiments performed with hexaethylstarch hemoglobin (HES). The results indicate that the calcium channel blocker (DLT) reduces the vasoconstrictive effect of PHE (a1, 0; b1, 10⁻⁷M; c1, 10⁻⁶M; d1, 10⁻⁵M). Similarly when HES (4.4 uM) is added to the tissue chambers with the same concentration of additives as in series (a), (see a2, b2, c2, d2) the vasoconstriction is reduced. Adding sildenafil citrate (10⁻⁵ M) to the tissue chambers, represented by e1 and e2, having the same additives as d1 and d2, the vasoconstriction is totally abolished.

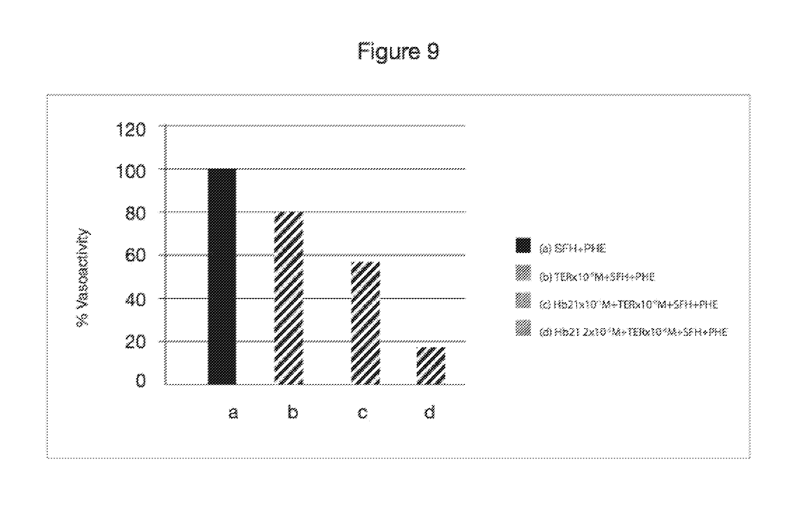

FIG. 9 illustrates a similar experiment conducted with an alpha agonist blocker, terazosin. The results indicate that terazosin in suboptimal concentration (10⁻⁸ M) reduces the vasoconstrictive effect of SFH. In combination with sildenafil citrate the vasoconstriction is reduced significantly. The bar graphs represent the following additives: (a) SFH+PHE, (b) TER+SFH+PHE, (c) TER+sildenafil citrate(10⁻⁵M)+SFH+PHE, (d) TER+sildenafil citrate (2×10⁵M)+SFH+PHE.

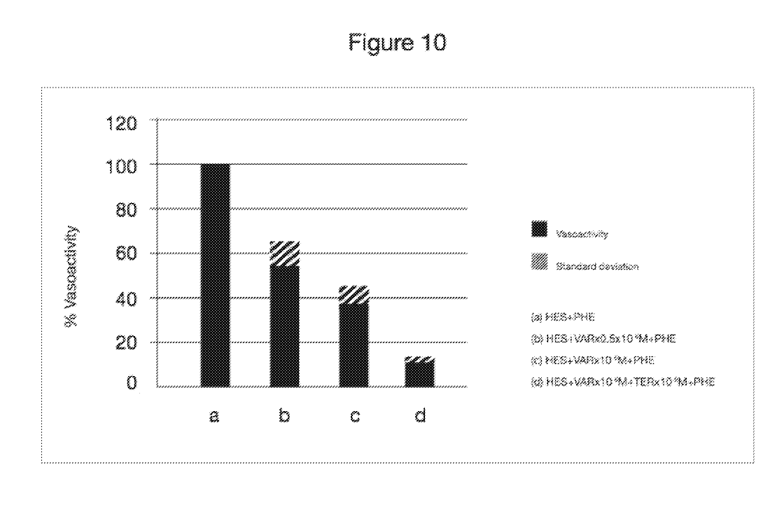

FIG. 10 illustrates the reduction of vasoconstriction by vardenafil and terazosin. In this graph HES (0.44 uM) was used in all the tissue chambers. The following additions were made in each of the four tissue chambers: (a) PHE 10⁻⁷ M, (b) VAR 0.5×10⁶ M+PHE 10⁻⁷ M, (c) VAR 10⁻⁶ M+PHE 10⁻⁷ M and (d) VAR 0.5×10⁻⁶ M+TER 10⁻⁸ M+PHE 10⁻⁷ M.

The above results indicate that both calcium channel blockers and alpha agonist blockers act synergistically with Hb21. The prevention of vasoconstriction, by these methods, imply that the smooth muscle contraction generated by the presence of SFH can be alleviated by a combination of suboptimal doses of calcium channel blockers, alpha adrenergic blockers and a PDE inhibitor, e.g. sildenafil citrate or vardenafil.

### Discussion

We have shown that SFH causes constriction of the smooth muscles in the segments of the rat thoracic aorta. This contraction depends on the presence of PHE. The clinical application of this phenomenon suggests the reason for increased cardiovascular and neurological side effects resulting from transfusions of stored blood and hemoglobin based blood substitutes.

In preliminary experiments it was also shown that SFH does not initiate vasoconstriction but addition of an alpha agonist (PHE) will increase the tension of the RTA significantly.

We have also shown above, that a PDE inhibitor, e.g. Hb21, will reduce the amplitude of the vasoconstriction. This implies that the cGMP generated prior to the addition of the SFH is preserved and can act to influence the PKG mechanism of smooth muscle relaxation or the PDE inhibitor allows by some undetermined mechanism to stimulate the guanylate cyclase.

To reduce the effective dose of Hb21 we have examined the effect of a calcium channel blocker and an alpha agonist blocker on the changes produced by Hb21 on the vasoactivity of RTA. We found that a combination of suboptimal doses of these agents and a suboptimal dose of a PDE inhibitor, e.g. sildenafil citrate and vardenafil, will abolish the vaso-constricting properties of SFH in the tissue chambers.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A process for reducing or preventing vasoactivity induced by the introduction of a composition containing a hemoglobin based oxygen carrier (HBOC), free hemoglobin (Hb), stored blood products having vasoactivity inducing concentrations of free tetrameric hemoglobin, and combinations thereof comprising:
combining at least one phosphodiesterase inhibitor (PDE) with at least one calcium channel blocker, and at least one alpha antagonist, with an HBOC or Hb containing material, in an amount effective to reduce or prevent said vasoactivity.

2. The process of claim 1 wherein said phosphodiesterase inhibitors are selected from the group consisting of 5-(2-Propoxyphenyl)-1H-[1,2,3]triazolo[4,5-d]pyrimidin-7(4H)-one, 2-o-propoxyphenyl-8-azapurine-6-one, 1-cyclopentyl-3-methyl-6-(4-pyridyl)pyrazolo[3,4-d]pyrimidin-4-(5H)-one, SCH 48936 ((+)−6a,7,8, 9,9a, 10,11,1 la-octahydro-2,5-dimethyl-3H-pentalen (6a,1,4,5)imidazo[2,1-b]purin-4(5H)-one, 2-phenyl-8-ethoxycycloheptimidazole, sodium 1-[6-chloro-4-(3,4-methylenedioxybenzyl)-aminoquinazolin-2-y]piperidine-4-carboxylate sesquihydrate, sildenafil, tadalafil, vardenafil, avanafil, lodenafil, mirodenafil, udenafil, zaprinast, xanthine, caffeine, theophylline, theobromine, aminophylline, oxtriphylline, dyphylline, pentoxifylline, isobutylmethyixanthine, dipyridamole, papaverine, and mixtures thereof.

3. The process of claim 1 wherein said calcium channel blockers are selected from the group consisting of amlodipine, diltiazem, felodipine, isradipine, nifedipine, nicardipine, nimodipine, nisoidipine, verapamil, and mixtures thereof.

4. . The process of claim 1 wherein said alpha agonists are selected from the group consisting of prazosin, terazosin, urapidil, labetalol, yohimbine, phenoxybenzamine, phentolamine, tolazoline, acebutolol, atenolol, and mixtures thereof.

5. A process for reducing vasoconstriction initiated by the adminsistration, to a patient, of a composition containing a hemoglobin based oxygen carrier (HBOC), free hemoglobin (Hb), stored blood products having vasoactivity inducing concentrations of free tetrameric hemoglobin, or combinations thereof, by administering, to said patient, a vasoconstriction reducing composition consisting of:
a vasoconstriction reducing amount of the phosphodiesterase inhibitor (PDE) sildenafil citrate to result in a final a concentration of between 10⁻⁴M and 10⁻⁵M in combination with vasoconstriction reducing amounts of the calcium channel blocker diltiazem to result in a final concentration of 10⁻⁵ M, and of the alpha antagonist terazosin to result in a final concentration between 10⁻⁵ M and 2×10⁻⁵ M,
whereby vasoconstriction initiated by the administration of a composition containing a hemoglobin based oxygen carrier (HBOC), free hemoglobin (Hb), stored blood products having vasoconstriction inducing concentrations of free tetrameric hemoglobin, or combinations thereof is reduced.

6-18. (canceled)

19. The process of claim 1 wherein said phosphodiesterase inhibitor is sildenafil citrate.

20. The process of claim 1 wherein said phosphodiesterase inhibitor is vardenafil.

21. The process of claim 1 wherein said calcium channel blocker is diltiazam.

22. The process of claim 1 wherein said alpha agonist is terazosin.

23. The process of claim 1 wherein the phosphodiesterase inhibitor is sildenafil citrate, and the calcium channel blocker is diltiazam.

24. The process of claim 1 wherein the phosphodiesterase inhibitor is sildenafil citrate, the calcium channel blocker is diltiazam, and the alpha agonist is terazosin.

25-30. (canceled)

31. A process for reducing or preventing vasoactivity induced by the introduction, into a patient in need thereof, of a composition containing a hemoglobin based oxygen carrier (HBOC), free hemoglobin (Hb), stored blood products having vasoactivity inducing concentrations of free tetrameric hemoglobin, and combinations thereof comprising:
providing, in combination, sildenafil citrate, a phosphodiesterase inhibitor, at a solution concentration of between 10⁻⁴ M and 10⁻⁵M, diltiazam, a calcium channel blocker, at a solution concentration of 10⁻⁵ M, and terazosin, an alpha antagonist, at a solution concentration between 10⁻⁵ M and 2×10⁻⁵M, and a solvent therefore;
whereby vasoactivity induced by the introduction of a composition containing a hemoglobin based oxygen carrier (HBOC), free hemoglobin (Hb), stored blood products having vasoactivity inducing concentrations of free tetrameric hemoglobin, and combinations thereof are reduced or prevented.
